(19) United States
(12) Patent Application Publication
(10) Pub. No.: US 2021/0000641 A1
(43) Pub. Date: Jan. 7, 2021

(54) EVA SEGMENTED INTRAVAGINAL RINGS CONTAINING PROGESTERONE

(71) Applicants: Bridget Martell, Guilford, CT (US); David Friend, San Deigo, CA (US); Martin Doorbar, Hampshire (GB); Shen Luk, Nottingham (GB)

(72) Inventors: Bridget Martell, Guilford, CT (US); David Friend, San Deigo, CA (US); Martin Doorbar, Hampshire (GB); Shen Luk, Nottingham (GB)

(21) Appl. No.: 16/835,282

(22) Filed: Mar. 30, 2020

Related U.S. Application Data
(60) Provisional application No. 62/826,978, filed on Mar. 29, 2019.
(60) Provisional application No. 62/843,288, filed on May 3, 2019.

(51) Int. Cl. A61F 6/08; A61L 31/04; A61L 31/16

(52) U.S. Cl. CPC A61F 6/08; A61L 2300/43; A61L 31/16; A61L 31/048

(57) ABSTRACT

Disclosed herein are segmented EVA Rings that contain progesterone that can be used to prevent preterm birth in subjects with a shortened cervix or to treat luteal phase deficiency or as luteal phase support.

### RELATED APPLICATIONS

This application claims the benefit of priority to U.S. Ser. No. 62/826,978, filed Mar. 29, 2019 and U.S. Ser. No. 62/843,288, filed May 3, 2019. The disclosures of which are incorporated by reference herein, including drawings.

### FIELD OF THE INVENTION

The present invention is in the field of intravaginal rings, in particular segmented, ethylene-vinyl acetate (EVA) intravaginal rings, and their use treating, ameliorating, or preventing, preterm births and for use in assisted reproduction technologies (ART).

### BACKGROUND OF THE DISCLOSURE

Preterm birth (defined as birth before 37 weeks of completed gestation) is the leading cause of neonatal deaths annually causing over 1 million deaths each year, which comprises 27% of 4 million neonatal deaths annually and 3.2 million stillbirths each year [1]. The need for new treatment strategies to reduce preterm birth and infant mortality was summarized by recently by the United States Centers for Disease Control and Prevention [2, 3]. Globally, the World Health Organization reports that 15 million preterm births occur each year. The data also suggest the rates of preterm birth are increasing in most countries and that prematurity is the second leading cause of death after pneumonia in children under the age of 5 [4].

A short cervix at mid-pregnancy is the most reliable predictor of preterm birth of a newborn. Children who are born preterm have a greater risk of chronic and life-threatening conditions at all stages of life. In infancy, these include respiratory distress syndrome, necrotizing enterocolitis, intraventricular hemorrhage, retinopathy of prematurity, and sepsis. In early childhood, they are at an increased risk for developmental delay, behavior and learning problems, and asthma. In adulthood, those who are born prematurely have an increased risk of arterial hypertension, diabetes mellitus, cardiovascular disease, and stroke [16-19]. Over a decade of evidence supports the use of vaginally administered P for the prevention of preterm birth in women having a short cervix at mid-pregnancy [8, 20, 9, 21, 22], yet there is still no FDA-approved progesterone product for this indication.

Progesterone (P) is a naturally occurring steroid that is secreted predominately by the ovary and placenta. In pregnancy, P helps the uterus grow [5, 6]. In many mammalian species, P plays a direct role in uterine quiescence (i.e., preventing labor), and the onset of labor is preceded by a decrease in P plasma concentrations. The role of P in the onset of human labor is less evident [7]. Although the precise mechanism(s) by which the mis-regulation of P activity leads to cervical shortening and preterm birth (PTB) are not known, there is evidence demonstrating the therapeutic efficacy of delivering supplemental, natural P directly to the cervix to extend gestation. Fonesca et al. demonstrated that daily, vaginal administration of 200 mg capsules of micronized P (Utrogestan®, Prometrium®) in women with a cervical length ≤15 mm is associated with a 44% reduction in the rate of PTB <34 weeks of gestation [8]. Similar findings were reported by DeFranco et al. [9]. Daily administration of micronized P gel (Crinone®) reduces the rate of PTB in women with a normal cervical length between 10 and 20 mm [10]. Further, P was associated with a significant reduction in the rate of respiratory distress syndrome. Neither Crinone or Prometrium are indicated for treatment of women to prevent preterm birth. A synthetic progesterone product, 17-alpha hydroxyprogesterone caproate (17-OHPC, Makena®) is indicated to reduce the risk of preterm birth in women with a singleton pregnancy who have a history of singleton spontaneous preterm birth; however, Makena is ineffective in preventing preterm birth in singleton pregnant women with short cervical length (SCL) [11, 12].

Despite the published evidence, medical guidelines, and consensus statements recommending the use of vaginal P for the prevention of preterm birth in women with SCL and singleton pregnancy, there is a paucity of data surrounding the optimal formulation and dosage of P. Systemic absorption of P from gel and capsule products has yet to be compared directly.

Vaginal progesterone is also being investigated for use as luteal phase support in in vitro fertilization although the dosage, timing, route of administration, timing for initiation of treatment, and duration of use is not well established (see Lockwood, et al., (2014), Fertility and Sterility, 102(1):112-119.

There have been a number of IVR formulations designed to release P over an extended period of time. An early example of a P-releasing IVR was a silicone reservoir device to be used by post-partum, lactating women for birth control [27]. These IVRs were designed to release about 5 mg per day for 90 days. More recently, another silicone IVR was developed and commercialized (Progering®, Grunenthal Laboratories) in several South American countries. Progering releases P at about 10 mg per day for 90 days [28-30]. Vaginal rings releasing P have also been investigated for luteal support during in vitro fertilization [31-35] although none have been approved for use by regulators.

Thus, there is an unmet need to develop P-releasing IVR rings for women at risk for preterm birth due to SCL, and for use in ART.

### SUMMARY OF THE INVENTION

Described herein are EVA-based segmented intravaginal rings (IVRs) which are designed to release about 4 to about 12 mg progesterone (P) per day over days 2-14.

In some embodiments, a rate of release of P is in the range of 4 mg/day to about 12 mg/day which includes ranges that fall in between the 4 mg/day to about 12 mg/day range. For example, without limitation, about 5 mg/day to about 12 mg/day, about 6 mg/day to about 8 mg/day, about 4 mg/day to about 7 mg/day, about 10 mg/day to about 12 mg/day etc. In some embodiments, the amounts the rate of release of P is an individual value in the range of 4 mg/day to about 12 mg/day, for example, about 5 mg/day, about 7 mg/day, about 9 mg/day etc.

An aspect of the disclosure is an ethylene-vinyl-acetate (EVA), intravaginal ring (IVR), where the ring contains at least two segments/fibers, wherein one segment contains progesterone (P) or P equivalent. In an embodiment of this aspect, the the ring is about 57 mm in diameter with a cross-section diameter of about 5 mm.

In one embodiment, the EVA ring releases about 4 mg/day P after day 1 of insertion.

In another embodiment, the EVA ring releases about 8 mg/day P after day 1 of insertion.

In another embodiment, the EVA ring releases about 12 mg/day P after day 1 of insertion.

In yet another aspect of the EVA ring, the EVA segment containing P is prepared using EVA (about 28% vinyl acetate content) with a final drug loading of about 27% w/w.

In some embodiments of the EVA ring, the EVA segment containing P is about 74.5 mm to about 156.0 mm in length or about 74.5mm to about 148.5 mm in length.

In some embodiments of the EVA ring, the length of the EVA segment containing P is selected from the size a size of about 74.5 mm, about 148.5 mm and about 156.0 mm.

In some embodiments, the EVA ring has a rate of release of P that is from about 4 mg/day, about 8 mg/day or about 12 mg/day days over a period of about 2 days through about 14 days.

In some embodiments of the EVA ring, the release rates for the 4 mg/day progesterone segmented IVR is about 20.5±2.2 for 0-24 hours; about 3.8±1.3 for days 2-14, and about 2.3±1.5 on day 14.

In some embodiments of the EVA ring, the release rates for the 8 mg/day progesterone segmented IVR is about 38.4±3.3 for 0-24 hours; about 7.4±2.4 for days 2-14, and about 4.7±1.9 on day 14.

In some embodiments of the EVA ring, the release rates for the 12 mg/day progesterone segmented IVR is about 60.5±2.5 for 0-24 hours; about 11.5±2.8 for days 2-14, and about 7.3±3.8 on day 14.

In some embodiments of the EVA ring, the Cₘₐₓ (pg/ml) for the 4 mg/day progesterone segmented IVR is about 969±145.

In some embodiments of the 4 mg/day P EVA ring, the AUC₀₋₃₃₆ₕ pg h/ml is about 153,000±38,900.

In some embodiments of the 4 mg/day P EVA ring, the C_{AVG} (pg/ml) is about 455±116.

In some embodiments of the 4 mg/day P EVA ring, the Tₘₐₓ (h) is about 12.

In some embodiments of the 8 mg/day P EVA ring, the Cₘₐₓ (pg/ml) is about 1,820±469.

In some embodiments of the 8 mg/day P EVA ring, the AUC₀₋₃₃₆ₕ is 229,000±40,700.

In some embodiments of the 8 mg/day P EVA ring, the C_{AVG} is about 682±121.

In some embodiments of the 8 mg/day P EVA ring, the Tₘₐₓ is about 2.

In some embodiments of the 12 mg/day P EVA ring, the Cₘₐₓ (pg/ml) is about 2,520±432.

In some embodiments of the 12 mg/day P EVA ring, the AUC₀₋₃₃₆ₕ (pg h/ml) is about 350,000±73,900.

In some embodiments of the 12 mg/day P EVA ring, the C_{AVG} (pg/ml) is about 1,040±220.

In some embodiments of the 12 mg/day P EVA ring, the Tₘₐₓ (h) is about 4.

In an aspect of the disclosure, the disclosed EVA rings are used to prevent preterm birth in which the subject being treated has a shortened cervix.

In another aspect of the disclosure, the disclosed EVA rings are used to treat or ameliorate luteal phase deficiency or as luteal phase support.

In another aspect of the disclosure, the EVA rings are used to provide progesterone for luteal phase support for women receiving assisted reproductive therapies.

In some embodiments, the disclosed EVA rings are used to treat or ameliorate luteal phase deficiency associated with low follicular-stimulating hormone (FSH) levels, altered FSH/luteinizing hormone (LH) ratio, or abnormal FSH and LH pulsatility.

In some embodiments, the luteal phase deficiency is caused by abnormal FSH or LH pulsatility is caused by hypothalamic amenorrhea, thyroid and prolactin disorders, obesity and polycystic ovary syndrome (PCOS) and controlled ovarian stimulation (COS) for IVF cycles.

In some embodiments, the disclosed rings are used to cause menstrual periods in women who have not reached menopause but are not having periods due to lack of progesterone in the body, or to prevent overgrowth in the lining of the uterus in postmenopausal women receiving estrogen replacement therapy.

### BRIEF DESCRIPTION OF THE DRAWINGS

FIG. 1 shows a schematic used to prepare the segmented EVA rings described herein.

FIG. 2. In vitro release of P from IVRs (4 mg/d, 8 mg/d, and 12 mg/d). Data are means ±SD (n=6).

FIG. 3. Plasma concentration-time profiles of P on Day 14 following once-daily dosing of Crinone 8% gel (90 mg P) and Prometrium 200 mg capsules. Data are mean ±SD (n=3).

FIG. 4. Plasma concentration-time profiles of P from Day 0 through Day 14 following a single administration of IVRs releasing P at 4 mg/d, 8 mg/d and 12 mg/d. Data are mean ±SD (n=5).

### DETAILED DESCRIPTION OF THE EMBODIMENTS

Disclosed herein are 14-day, segmented IVRs that deliver a relatively constant dose of P. The IVRs described herein are composed of EVA, and are formulated as matrix devices. The IVRs disclosed herein can be used to treat (e.g., cure, suppress), ameliorate, and/or prevent (e.g., delaying or preventing the onset, recurrence or relapse of) pre-term birth and can also be used as luteal phase support in in vitro fertilization.

The segmented rings described herein are advantageous as compared to other EVA rings systems in that the loading of segments can be optimized within a narrow range of EVA concentrations from about 27% to about 36%. This range, in about 28% vinyl acetate content ensures a stable system that avoids being at or around a transition point between fully dispersed drug in polymer and crystalline drug particles dispersed in polymer. The second advantage of the EVA ring systems described herein is the ability to easily change release rates through variations in segment length. Thus, preparation of clinical supplies for dose ranging studies is straight forward.

In some aspects of the invention described herein, where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. For example, a range from 27% to 36% would include 27% to 29%, or 27% to 33% or 33% to 35%, etc. Such ranges would also include individual points in the range, for example, 28%, 29%, 30% etc.

The disclosed IVRs herein are designed to have a rate of release of P from about 4 mg/day, about 8 mg/day and about 12 mg/day days over a period of about 2 through about 14 days, or a range of release of P from about 4 mg/day to about 12 mg/day.

In some embodiments, the release rates for the 4 mg/day progesterone segmented IVR is about 20.5±2.2 for 0-24 hours; about 3.8±1.3 for days 2-14 and about 2.3±1.5 on day 14.

In some embodiments the release rates for the 8 mg/day progesterone segmented IVR is about 38.4±3.3 for 0-24 hours; about 7.4±2.4 for days 2-14, and about 4.7±1.9 on day 14.

In some embodiments the release rates for the 12 mg/day progesterone segmented IVR is about 60.5±2.5 for 0-24 hours; about 11.5±2.8 for days 2-14, and about 7.3±3.8 on day 14.

In some embodiments, the Cₘₐₓ (pg/ml) for the 4 mg/day progesterone segmented IVR is about 969±145. The AUC₀₋₃₃₆ₕ pg h/ml is about 153,000±38,900. The C_{AVG} (pg/ml) is about 455±116 and Tₘₐₓ (h) is about 12.

In some embodiments, the Cₘₐₓ (pg/ml) for the 8 mg/day progesterone segmented IVR is about 1,820±469. The AUC₀₋₃₃₆ₕ (pg h/ml) is about 229,000±40,700. The C_{AVG} is about 682±121. The Tₘₐₓ (h) is about 2.

In some embodiments, the Cₘₐₓ (pg/ml) for the 12 mg/day progesterone segmented IVR is about 2,520±432. The AUC₀₋₃₃₆ₕ (pg h/ml) is about 350,000±73,900. The C_{AVG} (pg/ml) is about 1,040±220. The Tₘₐₓ (h) is about 4.

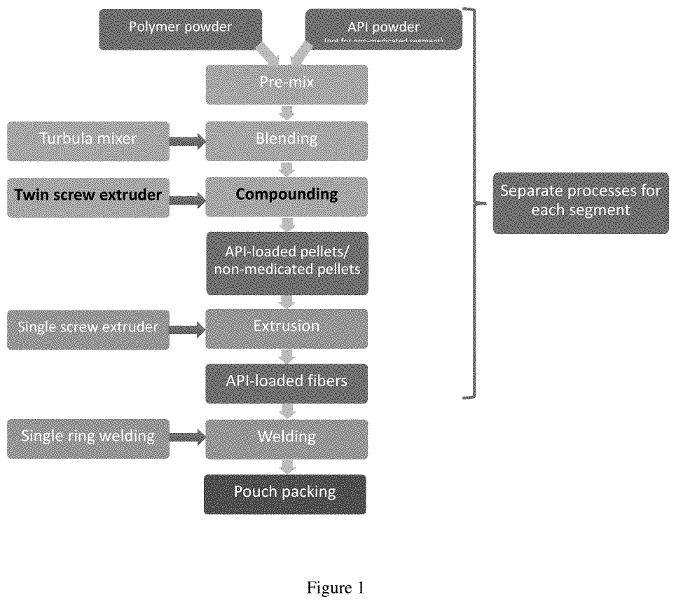

The disclosed IVRs are made by a process described in FIG. 1 and in a manner similar to that described previously [13]. All ring manufacturing took place at QPharma (Malmö, Sweden). The process comprised compounding pellets, extrusion of fibers followed by joining of the fibers by heat welding. Blending was accomplished using a Turbula mixer (Model T 10 B, with a 17-liter stainless steel mixing vessel, Glenn Mills, Clifton, N.J.). The resulting blend was then compounded by hot-melt extrusion using a twin-screw extruder (Pharma 11 Twin Screw Hot Melt Extruder with a Pharma 11 gravimetric feeder) and fed onto a Pharma 11 Air Cooled conveyor followed by pelletization using a Pharma 11 Vericut Pelletizor (Thermo Fisher Scientific, Dreieich, Germany). The pellets were formed into fibers by hot melt extrusion using a 25 mm single screw extruder (Dr Collin, Ebersberg, Germany). The resulting fibers were cut using a Dr Collin in-line Cutting Station. Cut fibers (or segments) were welded using Automationspartner single station laboratory welder (Ramlösa, Sweden).

In some embodiments, the segments containing progesterone of the IVRs disclosed herein are in the range of about 74.5 mm to about 156.0 mm. In some embodiments the segments are in the range of about 74.5 mm to about 148.5 mm. In some embodiments the segments of the IVRs disclosed herein are about 74.5 mm, about 148.5 mm or about 156 mm. In some embodiments the segments contain about 5 mg to about 10 mgs progesterone or progesterone analog. In some embodiments the segments contain about 27 w/w progesterone.

In some embodiments, the IVRs are about 57 mm in diameter with a cross-section diameter of about 5 mm.

The intravaginal rings disclosed herein were designed to release about 4, 8, or 12 mg P/per day over days 2 through 14. The rings evaluated herein, including placebo IVRs, were administered to drug-naïve ovariectomized female Dorset crossbred sheep for 14 days after which they were removed and a new P-releasing IVR was administered for 14 additional days. For comparison purposes, a group received Crinone 8% gel (approximately 90 mg P) and a second group received Prometrium (200 mg P) capsules intravaginally on a once-daily basis. Data collected included plasma pharmacokinetics of P and microscopic assessment of the uterus, cervix, and vagina.

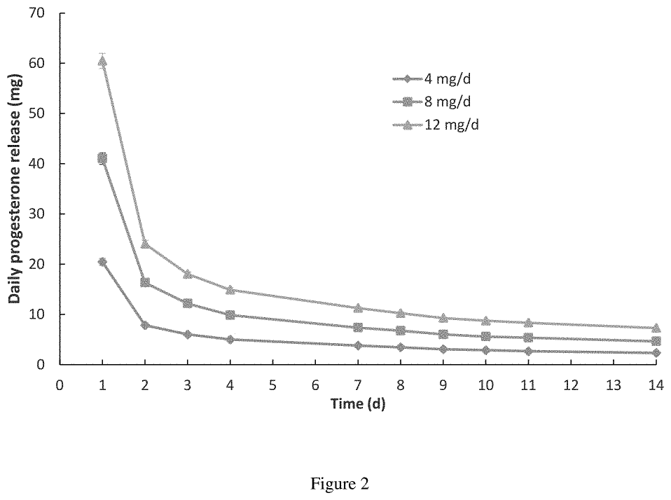

Release rates of P over the 14-day test period are characterized by the rate over Day 1, from Day 2 to Day 14, and the rate on Day 14. Table 1 shows the data collected in this manner from the different P-releasing IVRs. The release rate of P from Day 2 through 14 were close to the target release rates of 4 mg/d (3.8±1.3 mg/d), 8 mg/d (7.4±3.3 mg/d), or 12 mg/d (11.5±2.8 mg/d). The in vitro release profile is typical of a matrix-type delivery system with a relatively rapid release of drug followed by a period of slower release which can be seen in FIG. 2

TABLE 1
Release rates (mg/d) from the 4 mg/d, 8 mg/d, and 12 mg/d P IVRs

| | | 4 mg/d IVR | 8 mg/d IVR | 12 mg/d IVR |
|---|---|---|---|---|
| Test Time Point(s) | | (mg/d) | (mg/d) | (mg/d) |
| 0-24 | h | 20.5 ± 2.2^{a} | 38.4 ± 3.3 | 60.5 ± 2.5 |
| 2-14 | d | 3.8 ± 1.3 | 7.4 ± 2.4 | 11.5 ± 2.8 |
| 14 | d | 2.3 ± 1.5 | 4.7 ± 1.9 | 7.3 ± 3.8 |

All data are means ± SD (n = 6)

Following administration of the IVRs, daily vaginal ring checks confirmed the IVRs remained in place until the time of removal on Day 15 and Day 29. All rings remained in place for their appropriate duration with the exception of one animal in Group 5 (8 mg/d) on Day 16 when it was apparently expelled and could not be located. Therefore, a new ring was inserted in this animal on Day 16. Additionally, on Day 21 a ring from one animal in Group 6 (12 mg/d) was found in the bedding. The ring was visually inspected for damage, cleaned with lukewarm water, dried, and reinserted.

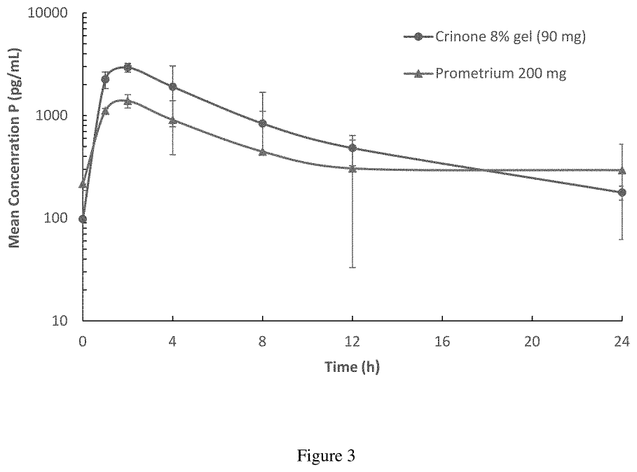

The plasma concentration vs. time curves for P for Groups 1 and 2 indicate a somewhat rapid absorption process (Tₘₐₓ of 2 hours) and first order elimination with a 10-fold range of concentrations during a steady-state dosing interval (FIG. 3). The PK parameters on Day 14 of once-daily vaginal administration of Crinone 8% gel and Prometrium 200 mg capsules are summarized in Table 2. Despite approximately half of the dose administered as Crinone 8% gel compared with Prometrium (90 mg vs. 200 mg), Cₘₐₓ, and AUC₀₋₂₄ₕ were substantially higher indicating a greater rate and extent of absorption from Crinone.

TABLE 2
PK parameters from Crinone 8% gel (90
mg) and Prometrium 200 mg capsules

| | | |
|---|---|---|
| PK | Group 1 | Group 2 |
| Parameter | 90 mg P^{a} | 200 mg P^{b} |

| | | | |
|---|---|---|---|
| Cₘₐₓ | (pg/mL) | 3,020 ± 140 | 1,390 ± 206 |
| AUC_{0-24 h} | (h*pg/mL) | 20,700 ± 1,640 | 12,000 ± 4,090 |
| C_{AVG} | (pg/mL) | 863 ± 68.5^{c} | 501 ± 170 |
| Tₘₐₓ | (h) | 2 (2-4)^{d} | 2 (2-2)^{d} |

^{a}90 mg dose of P is approximately 1.5 mg/kg based on a sheep of 60 kg
^{b}200 mg dose of P is approximately 3.3 mg/kg based on a sheep of 60 kg
^{c}C_{AVG} = AUC_{0-24 h}/24 h
^{d}Median (minimum-maximum), median value only reported if actual collection interval

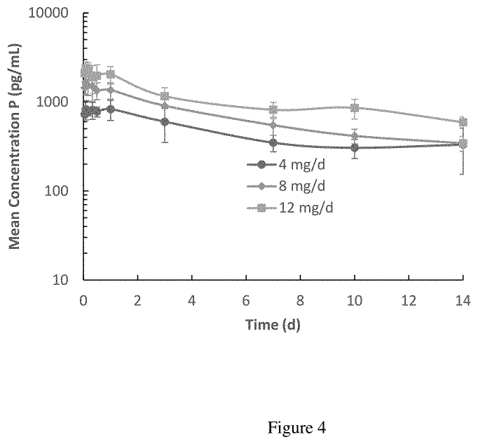

The concentration over time for the 4, 8, and 12 mg/d IVR groups (FIG. 4) generally showed a sustained release profile. This indicates a slow release and absorption process, the rate-determining step in the prolonged 14-day profile. Table 3 shows the PK parameters following administration on Day 1 and followed for 14 days. In general, the PK parameters from the three different IVR increased with increasing release rate. While not directly comparable, the PK parameters from the 12 mg/d IVRs were most similar to those from Crinone 8% vaginal gel. Following removal of the IVRs on Day 29 (the second ring administered), the rings were analyzed for residual P. The amounts released were calculated by adding the amount of P measured in the rings after 14 days of use in sheep to the cumulative amount released over 14 days under in vitro dissolution, and then dividing by the content as measured by assay. Doing so showed that all rings were within ±10% of mass balance. There was consistency between in vitro dissolution and in vivo release as well. For instance, the 8 mg/d IVRs had an initial average loading of 14 mg P; following 14 days of release in the animals, the residual mean amount of P remaining was 387±34 mg (n=5) or 130 mg P released over 14 days. In vitro, the cumulative amount of P release over 14 days was 139 mg. Similar results were found for IVRs releasing P at 4 and 12 mg/d.

TABLE 3
Pharmacokinetic parameters of P from intravaginal rings

| PK | | Group 4 | Group 5 | Group 6 |
|---|---|---|---|---|
| Parameter | | 4 mg/d | 8 mg/d | 12 mg/d |
| Cₘₐₓ | (pg/mL) | 969 ± 145 | 1,820 ± 469 | 2,520 ± 432 |
| AUC_{0-336 h} | (pg*h/mL) | 153,000 ± 38,900 | 229,000 ± 40,700 | 350,000 ± 73,900 |
| C_{AVG} | (pg/mL) | 455 ± 116^{a} | 682 ± 121 | 1,040 ± 220 |
| Tₘₐₓ | (h) | 12 (1-72)^{b} | 2 (1-8)^{b} | 4 (2-8)^{b} |

^{a}C_{AVG} = AUC_{0-14 d}/14 d
^{b}Median (minimum-maximum), median value only reported if actual collection interval

Animal evaluations conducted during the treatment period were unremarkable for abnormal physical findings. Enlarged udders were observed in several animals (including some animals with enlargement prior to administration of study treatment), but since all animals had a history of lactation, these observations were not considered to be related to the study treatment. One Group 2 animal was observed with red coloration of the vulva during Week 4, which correlated with vaginal irritation findings (see below). A clear vaginal discharge was observed in two animals from Group 4 during Weeks 3 and Week 3 and 4, respectively. Discharge findings are common during studies with the intravaginal route.

Evaluations of external vaginal irritation performed on Days 1 through 29 of the treatment-period comprised assessments of erythema and edema on a scale of 0 to 4 (Table 4). The number of incidences of very slight erythema and eschar was slightly elevated for animals administered the highest IVR dose (12 mg/d); however, the irritation was limited to two of the five animals in the treatment group. In all other treatment groups, very slight erythema was observed in only one animal on two occasions or in two animals on one occasion. Very slight edema was observed on no more than one occasion/animal in the Crinone 8% gel and placebo and 12 mg/d IVR-treated groups. All external vaginal irritation findings were transient and were no longer present by study termination. The findings were most commonly observed after the first insertion of the IVR-treated groups and during the first 14 days of study for the Crinone 8% gel treated group.

TABLE 4
External and internal vaginal irritation scoring

| Treatment^{a} | | | | | |
|---|---|---|---|---|---|
| Group 1 | Group 2 | Group 3 | Group 4 | Group 5 | Group 6 |

External irritation, Days 1 through 29

| | | | | | | |
|---|---|---|---|---|---|---|
| Erythema and | | | | | | |
| eschar score | | | | | | |
| 0 | 76 | 85 | 85 | 143 | 143 | 135 |
| 1 | 11 | 2 | 2 | 2 | 2 | 10 |
| 2 | 0 | 0 | 0 | 0 | 0 | 0 |
| 3 | 0 | 0 | 0 | 0 | 0 | 0 |
| 4 | 0 | 0 | 0 | 0 | 0 | 0 |
| Edema score | | | | | | |
| 0 | 86 | 87 | 86 | 145 | 145 | 144 |
| 1 | 1 | 0 | 1 | 0 | 0 | 1 |
| 2 | 0 | 0 | 0 | 0 | 0 | 0 |
| 3 | 0 | 0 | 0 | 0 | 0 | 0 |
| 4 | 0 | 0 | 0 | 0 | 0 | 0 |

Internal irritation, Day 29

| | | | | | | |
|---|---|---|---|---|---|---|
| Erythema and | | | | | | |
| eschar score | | | | | | |
| 0 | 3 | 3 | 3 | 0 | 1 | 3 |
| 1 | 0 | 0 | 0 | 3 | 4 | 1 |
| 2 | 0 | 0 | 0 | 2 | 0 | 1 |
| 3 | 0 | 0 | 0 | 0 | 0 | 0 |
| 4 | 0 | 0 | 0 | 0 | 0 | 0 |
| Edema score | | | | | | |
| 0 | 3 | 3 | 3 | 5 | 5 | 5 |
| 1 | 0 | 0 | 0 | 0 | 0 | 0 |
| 2 | 0 | 0 | 0 | 0 | 0 | 0 |
| 3 | 0 | 0 | 0 | 0 | 0 | 0 |
| 4 | 0 | 0 | 0 | 0 | 0 | 0 |

^{a}Total number of observations made in Groups 1 through 3: external irritation = 87 (n = 3) and 145 in Groups 4 through 6 (n = 5)

Incidence counts of internal erythema and edema scores are also summarized in Table 4. The number of incidences of very slight and well-defined erythema was slightly elevated for animals administered the low dose IVR (4 mg/d). Very slight erythema was observed in 4 of 5 animals in the mid dose of IVR (8 mg/d). The high dose IVR group (12 mg/d) had the lowest number of incidences (2 of 5 animals) with one animal observed with very slight and one animal observed with well-defined erythema. In all other treatment groups, no erythema was observed in any animal. Due to the lack of a dose response and lack of a microscopic correlate, these findings were not considered to be IVR-related.

IVR-related microscopic observations indicated that the uterus, cervix, and vagina were generally small and inactive in all animals, which was considered a normal feature in ovariectomized animals Table 5. There was a minimal to mild mononuclear cell infiltration present within the vagina of all animals including the placebo controls, which was often accompanied by a minimal to mild neutrophilic infiltration. Minimal focal epithelial degeneration was present within the vagina of two individual animals, including placebo controls. Due to the lack of a dose response and similarities across the groups including the placebo IVRs, these changes were not considered to be IVR-related.

Assessment of the vaginal irritation index according to the method of Eckstein (Table 6) showed minimal irritation in all treatment groups, with irritation scores typically highest in the cranial portion of the vagina where the IVR was placed. Median scores in all groups were within the minimal irritation category (defined as scores of 1-4).

TABLE 5
Histologic findings of uterus, cervix, and vagina (cranial, mid,
and uro) following administration of placebo and active IVRs

| | | | Group 3 | Group 4 | Group 5 | Group 6 |
|---|---|---|---|---|---|---|
| | | | Treatment: | | | |
| | | | Placebo | 4 mg/d | 8 mg/d | 12 mg/d |
| | | | IVR | IVR | IVR | IVR |
| | | | Number examined | | | |
| | | | 3 | 5 | 5 | 5 |
| Uterus | Cyst | minimal | 0 | 0 | 1 | 0 |
| Cervix | No visible | | 3 | 5 | 5 | 5 |
| | lesions | | | | | |
| Vagina, | Infiltration | minimal | 3 | 2 | 3 | 4 |
| Cranial | neutrophil | mild | 0 | 0 | 0 | 1 |
| | Infiltration, | minimal | 3 | 5 | 5 | 5 |
| | mononuclear | | | | | |
| | cell | | | | | |
| | Degeneration | minimal | 1 | 1 | 0 | 0 |
| Vagina, | Infiltration | minimal | 2 | 2 | 2 | 5 |
| Mid | neutrophil | | | | | |
| | Infiltration, | minimal | 3 | 4 | 5 | 4 |
| | mononuclear | mild | 0 | 1 | 0 | 1 |
| | cell | | | | | |
| | Dilation, | minimal | 0 | 0 | 1 | 0 |
| | gland/lumen | | | | | |
| Vagina, | Infiltration | minimal | 2 | 3 | 3 | 4 |
| Uro | neutrophil | mild | 1 | 0 | 0 | 0 |
| | Infiltration, | minimal | 3 | 5 | 4 | 4 |
| | mononuclear | | | | | |
| | cell | | | | | |
| | Degeneration | minimal | 0 | 1 | 0 | 0 |
| | Dilation, | minimal | 0 | 1 | 0 | 0 |
| | gland/lumen | | | | | |
| | Dilation, | minimal | 0 | 1 | 0 | 0 |
| | gland/lumen | | | | | |

TABLE 6
Collective group mean IVR vaginal irritation scores

Treatment

| | Placebo | 4 mg/d | 8 mg/d | 12 mg/d |
|---|---|---|---|---|
| Vaginal | (Group 3) | (Group 4) | (Group 5) | (Group 6) |
| Location | n = 3 | n = 5 | n = 5 | n = 5 |
| Cranial | 1.67 | −0.47 | −0.67 | −0.47 |
| Mid | 1.00 | 0.20 | 0.00 | 0.20 |
| Uro | 1.33 | −0.13 | −0.33 | −0.53 |

### Uses

The EVA rings disclosed herein can be used to cause menstrual periods in women who have not yet reached menopause but are not having periods due to a lack of progesterone in the body.

The EVA rings disclosed herein can be used with fertility treatment as part of Assisted Reproductive Technology (ART) as luteal phase support.

The EVA rings disclosed herein can be used to prevent overgrowth in the lining of the uterus in postmenopausal women who are receiving estrogen hormone replacement therapy.

The EVA rings disclosed herein can be used to treat luteal phase deficiency such as associated with low follicular-stimulating hormone (FSH) levels, altered follicular FSH/luteinizing hormone (LH) ratio and/or abnormal FSH and LH pulsatility, such as functional hypothalamic amenorrhea, thyroid and prolactin disorders, obesity and polycystic ovary syndrome (PCOS) and during controlled ovary stimulation (COS) for IVF cycles Palomaba et al., (2015) J Ovarian Res., 8:77.

### EXAMPLES

### Intravaginal Rings

Intravaginal rings capable of releasing P were prepared in a manner similar to that described previously [13]. The overall process is shown in FIG. 1. All ring manufacturing took place at QPharma (Malmö, Sweden). The process involved compounding pellets, extrusion of fibers followed by joining of the fibers by heat welding. Blending was accomplished using a Turbula mixer (Model T 10 B, with a 17-liter stainless steel mixing vessel, Glenn Mills, Clifton, N.J.). The resulting blend was then compounded by hot-melt extrusion using a twin-screw extruder (Pharma 11 Twin Screw Hot Melt Extruder with a Pharma 11 gravimetric feeder) and fed onto a Pharma 11 Air Cooled conveyor followed by pelletization using a Pharma 11 Vericut Pelletizor (Thermo Fisher Scientific, Dreieich, Germany). The pellets were formed into fibers by hot melt extrusion using a 25 mm single screw extruder (Dr Collin, Ebersberg, Germany). The resulting fibers were cut using a Dr Collin in-line Cutting Station. Cut fibers (or segments) were welded using Automationspartner single station laboratory welder (Ramlösa, Sweden).

IVRs capable of releasing P (EP, micronized, Pfizer, Inc. Kalamazoo, Mich.) at the desired rates were prepared by using fibers of varying length and drug loading. All IVRs were 57 mm in overall diameter with a cross sectional diameter of 5 mm. IVRs releasing 4 and 8 mg/d were prepared using EVA (28% vinyl acetate content, Vitaldose®, Celanese Corporation, Boucherville, Canada; or Polysciences Inc, Warrington, Pa.) with a final drug loading of 27% w/w. To create the 4 mg/d ring, the drug-containing segment length was 50 mm with a placebo segment length of 113.5 mm. The 8 mg/d IVR was created with a 100 mm drug-containing segment and a placebo segment of 63.5 mm. The 12 mg/d IVRs were prepared with segments loaded with 36% P (w/w) with a drug containing segment of 148.5 mm and a placebo segment of 15 mm. Placebo IVRs were prepared by welding three, drug free segments of 74.0, 74.5, and 15 mm. Each IVR weighs approximately 3 g.

### In vitro Release of P from IVRs

Before conducting the sheep study, the release rates of P from the three IVR formulations were measured in vitro to determine whether the target release rates had been attained. Release rates were tested using 200 mL 0.5% sodium dodecyl sulfate as a release medium, in shakers at 37° C. Sampling (2 mL) was conducted at 6 h, days 1-4, 7-11, 14, 15, 18, 21, 22, 25, and 28. Concentrations of P were determined using a validated reverse-phase liquid chromatography method using UV detection. The column used was a Phenomenex Luna C8(2), 150 mm×3.0 mm, 5 μm. The guard column used was Phenomenex C8 (4 mm×3 mm). The mobile phase was acetonitrile 45% in purified water (55%), v/v. The injection volume was 10 μL. P was detected by UV at 245 nm. The standard curve range for P was 0.00625-0.25 mg/mL. Over these concentrations the curve was linear (correlation coefficient >0.997). Six IVRs were tested at each dissolution time point.

### Sheep Study

The purpose of this study was to evaluate the in vivo PK and local tolerability of P-releasing and IVRs in drug-naïve ovariectomized female Dorset crossbred sheep and to compare with vaginally administered Crinone 8% gel (1.125 g or 90 mg P; Actavis Pharma, Parsippany, N.J.) and Prometrium 200 mg capsules (Solvay Pharmaceuticals, Marietta, Ga.). Data from the placebo IVR group were also collected as a comparison.

The animal study was conducted by an American Association for Accreditation of Laboratory Animal Care (AAALAC) accredited contract research organization facility (MPI Research, a Charles River Company, Mattawan, Mich.). The study was conducted in compliance with the US Food and Drug Administration (FDA) Good Laboratory Practices (GLP) Regulations and the US Department of Agriculture (USDA) Animal Welfare Act. A total of 27 experimentally naïve, female, uniparous, Dorset crossbred sheep, approximately 15.5 to 19 months of age at receipt, were received from Lauwers Lamb, Capac, Mich. Animals were identified by implanted microchips and by individual ear tags.

During acclimation, the animals were observed daily with respect to general health and any signs of disease. All animals were given a detailed clinical examination, and body weights were recorded within 3 days of receipt and again prior to the operating procedures. All animals were negative for Cryptosporidium and Giardia species. Strongyloides and Coccidia were detected in stool samples from almost all animals. Animals were treated with a single administration of fenbendazole (10 mg/kg orally). Animals weighed 57.5 to 77.0 kg at randomization.

Between 26 and 54 days before the scheduled dosing, all animals underwent a surgical procedure to remove the ovaries, in accordance with the research facility's standard operating procedures. Animals were allowed to recover for 26 to 54 days prior to dosing. During this recovery period, body weight measurements and clinical observations were performed weekly. Ovariectomy surgery was performed successfully in all animals as determined by undetectable levels of endogenous hormones. During the study, all animals were observed twice daily for morbidity, mortality, injury, and the availability of food and water. Detailed examinations of each animal were performed weekly during the study. These observations included evaluation of the skin, fur, eyes, ears, nose, oral cavity, thorax, abdomen, external genitalia, limbs and feet, respiratory and circulatory effects, autonomic effects such as salivation, and nervous system effects including tremors, convulsions, reactivity to handling, and unusual behavior.

Animals were randomly allocated to one of six treatment groups: Group 1 (n=3) Crinone 8% gel; Group 2 (n=3) Prometrium 200 mg capsules; Group 3 (n=3) placebo IVRs; Group 4 (n=5) 4 mg/d IVRs; Group 5 (n=5) 8 mg/d IVRs; or Group 6 (n=5) 12 mg/d IVRs.

The IVRs were stored at 2 to 8° C. until use and were allowed to warm to room temperature for 30 to 120 minutes prior to administration; Crinone 8% gel and Prometrium 200 mg capsules were maintained at room temperature in accordance with their labels. Both Crinone 8% gel and Prometrium 200 mg capsules were administered on Day 1 and every day thereafter ±2 h through Day 28. All IVRs were inserted on Day 1 remained in place through Day 14; the rings were then removed and a new ring inserted on Day 15. The second ring remained in place until Day 29 at which time it was removed. Vaginal ring insertion was performed as a clean procedure. The IVR was photographed before being digitally inserted into the cranial vagina using a gloved finger. During the treatment period, animals were digitally examined daily to confirm that the IVR was still in place.

Following completion of treatment on Day 29, the IVRs were removed from each animal and were photographed and stored at 2 to 8° C. before being returned for analysis of residual P content. Residual P in each IVR was performed as follows. Each IVR was cut into 2-3 segments then placed in a 200 mL volumetric flask. Tetrahydrofuran (100 mL at 37° C.) was added to each flask containing the ring segments and shaken at about 180 rpm for 2 h. Following dissolution of the IVRs, the EVA was precipitated by addition of methanol (90 mL). The resulting solution was passed through a 0.45 μm filter. The filtrate (5 mL) was diluted with 45 mL acetonitrile/H2O (70:30). The amount of P was determined using a validated HPLC method using UV detection at 220 nm. The theoretical mass balance was calculated by adding the amount of P present in the device after use with the amount released by the IVR (taken from the initial release testing of the IVRs in vitro) and dividing by the theoretical drug content. The amount of P remaining in the IVRs following dosing was determined as a gross check on IVR performance, and results were not intended to be correlated with PK findings.

### Pharmacokinetics (PK)

Blood samples for determination of the plasma concentrations of P (as applicable) were collected from all animals; animals were not fasted prior to blood collections. Samples from Groups 1 through 6 were collected prior to ovariectomy surgery, on Day −14 (to confirm successful ovariectomy). In Groups 1 and 2, plasma was collected on Day 14 pre-dose and again on day 7 pre-dose and at 1, 2, 4, 8, 12, and 24 h post-dose. In Group 3, plasma was collected Day 1 pre-dose and at 1, 4, 336, and 672 h post-dose. In Groups 4, 5, and 6, plasma was collected prior to insertion of the IVR (0 hours), and at 2, 4, 8, 12, 24, 48, 72, 168, 240, 336 h (prior to removal) post-dose.

Blood samples were placed in tubes containing K₂-EDTA and were centrifuged under refrigerated conditions within 60 minutes of sample collection. The resulting plasma was stored frozen at −60 to −90° C. within 120 minutes of sample collection. Plasma samples were shipped on dry ice for analysis (Pyxant Labs, Inc., Colorado Springs, Colo.). Plasma samples were analyzed using a liquid chromatography-mass spectrometry/mass spectrometry method validated according to bioanalytical method guidelines. The standard curve range for P was 0.1-20 ng/mL. Based on quality control samples, accuracy ranged from 96.5-98.0% for P. Precision (% CV) was less than 7.5% for P. The lower limit of quantitation (LLOQ) was 0.1 ng/mL with an upper limit of quantitation of 20 ng/mL. Concentrations below the LLOQ were set to zero for PK analyses.

Standard noncompartmental PK analysis methods were used. The area under the concentration-time curve (AUC) values were estimated by the trapezoidal rule. The C_{avg} (defined as average plasma concentration over the entire dosing interval, calculated as AUC_{TAU}/dosing interval where TAU is the dosing interval for steady-state data) was determined following administration of Crinone 8% gel and Prometrium 200 mg capsules (AUC_{0-24 h}/24 h) or IVR administration calculated as AUC_{0-336 h}/336 h.

### Tolerability: Description of Assessments, Grading Scale

The external vagina (the vulva and the externally visible portion of the vestibule) of all animals was examined prior to administration of all test articles and daily examinations were conducted on Days 2 through 29, prior to the daily ring checks or product administration. The external vagina was observed for gross signs of irritation (i.e., erythema and edema) and any other signs of local or systemic effect. Irritation was scored based on the Draize scale [14]; erythema and edema formation were rated on a scale of 0 (none) to 4 (severe). The same scales were used at necropsy on Day 29 to score irritation of the internal vagina (the portion not visible during in-life assessments); any other signs of local or systemic effects were also recorded.

### Necropsy

On Day 29, following external vaginal irritation scoring and ring removal, animals in all Groups were euthanized. At necropsy, a macroscopic examination of the reproductive organs and surrounding tissues was performed and the uterus, cervix, and vagina were collected and fixed in 10% neutral buffered formalin. Microscopic examination of reproductive tissues was conducted routinely processed hematoxylin and eosin-stained slides by a board-certified veterinary pathologist (J.D.V).

Vaginal irritation was scored based on the rabbit vaginal irritation method described by Eckstein et al. [15]. For each animal, 3 vaginal regions including the portion adjacent to the cervix (cranial), the middle portion (mid), and the portion at the level of the urethra (uro) were scored separately for 4 parameters (epithelial damage, vascular congestion, edema, and leukocyte infiltration) with each parameter receiving a score of 0 (normal) to 4 (marked). An overall vaginal irritation score was calculated for each vaginal region for each group by taking the sum of scores for all 4 parameters per site, dividing by the number of animals, and subtracting the average for the placebo control group from a companion study under similar conditions. Therefore, the overall vaginal irritation score ranged from 0 to a maximum of 16.

### Statistical Analyses

Statistical analysis of data was limited to calculation of descriptive statistics, including means, standard deviations (SD), group size for each group and time period (continuous endpoints), and either medians or incident counts for each group and time period (categorical endpoints).

### REFERENCES

1. Lawn J E, Gravett M G, Nunes T M, Rubens C E, Stanton C, Group tGR. Global report on preterm birth and stillbirth (1 of 7): definitions, description of the burden and opportunities to improve data. BMC Pregnancy and Childbirth. 2010;10(1):S1. doi:10.1186/1471-2393-10-s1-s1.
2. Preterm birth. Centers for Disease Control and Prevention. 2016. Accessed 13 Aug. 2018 2018.
3. Infant Mortality. Centers for Disease Control and Prevention. 2016. Accessed 13 Aug. 2018 2018.
4. March of Dimes. Save the Childre. Born Too Soon: The Global Action Report on Preterm Birth. In: Howson C, Kinney M, Lawn J, editors. Geneva: WHO; 2012.
5. Blanks A M, Brosens J J. Progesterone action in the myometrium and decidua in preterm birth. Facts Views Vis Obgyn. 2012;4(3):33-43.
6. Gellersen B, Fernandes M S, Brosens J J. Non-genomic progesterone actions in female reproduction. Human Reproduction Update. 2009;15(1):119-38. doi:10.1093/humupd/dmn044.
7. Caritis S N, Feghali M N, Grobman W A, Rouse D J. What we have learned about the role of 17-alpha-hydroxyprogesterone caproate in the prevention of preterm birth. Seminars in Perinatology. 2016;40(5):273-80.
8. Fonseca E B, Celik E, Parra M, Singh M, Nicolaides K H. Progesterone and the Risk of Preterm Birth among Women with a Short Cervix. New England Journal of Medicine. 2007;357(5):462-9. doi:10.1056/NEJMoa067815.
9. De Franco E A, O'Brien J M, Adair C D, Lewis D F, Hall D R, Fusey S et al. Vaginal progesterone is associated with a decrease in risk for early preterm birth and improved neonatal outcome in women with a short cervix: a secondary analysis from a randomized, double-blind, placebo-controlled trial. Ultrasound in Obstetrics & Gynecology. 2007;30(5):697-705. doi:doi:10.1002/uog.5159.
10. Hassan S S, Romero R, Vidyadhari D, al. e. Vaginal progesterone reduces the rate of preterm birth with sonographic short cervix: a multicenter, randomized, double-blind, placebo-controlled trial. Ultrasound in Obstetrics & Gynecology. 2011;38(1):18-31. doi:doi:10.1002/uog.9017.
11. Winer N, Bretelle F, Senat M-V, Bohec C, Deruelle P, Perrotin F et al. 17 alpha-hydroxyprogesterone caproate does not prolong pregnancy or reduce the rate of preterm birth in women at high risk for preterm delivery and a short cervix: a randomized controlled trial. American Journal of Obstetrics & Gynecology. 2015;212(4):485.e1-.e10. doi:10.1016/j.ajog.2014.10.1097.
12. Grobman W A, Thom E A, Spong C Y, Iams J D, Saade G R, Mercer B M et al. 17 alpha-hydroxyprogesterone caproate to prevent prematurity in nulliparas with cervical length less than 30 mm. American Journal of Obstetrics & Gynecology. 2012;207(5):390.e1-.e8. doi:10.1016/j.ajog.2012.09.013.
13. Kimball A B, Javorsky E, Ron E S, Crowley W, Langer R. A novel approach to administration of peptides in women: Systemic absorption of a GnRH agonist via transvaginal ring delivery system. Journal of Controlled Release. 2016;233:19-28.
14. Draize J H, Woodard G, Calvery H O. Methods for the study of irritation and toxicity of substances applied topically to the skin and mucous membranes. Journal of Pharmacology and Experimental Therapeutics. 1944;82(3):377-90.
15. Eckstein P, Jackson M C N, Millman N, Sobrero A J. Comparison of vaginal tolerance tests of spericidal preparations in rabbits and monkeys. Journal of Reproduction and Fertility. 1969;20(1):85-93. doi:10.1530/jrf.0.0200085.
16. Blencowe H, Cousens S, Chou D, Oestergaard M, Say L, Moller A-B et al. Born Too Soon: The global epidemiology of 15 million preterm births. Reproductive Health. 2013;10(1):52. doi:10.1186/1742-4755-10-s1-s2.
17. Sipola-Leppänen M, Vääräsmäki M, Tikanmäki M, Matinolli H-M, Miettola S, Hovi P et al. Cardiometabolic Risk Factors in Young Adults Who Were Born Preterm. American Journal of Epidemiology. 2015;181(11):861-73. doi:10.1093/aje/kwu443.
18 Tikanmäki M, Tammelin T, Sipola-Leppänen M, Kaseva N, Matinolli H-M, Miettola S et al. Physical Fitness in Young Adults Born Preterm. Pediatrics. 2016;137(1). doi:10.1542/peds.2015-1289.
19. Norman M. Preterm birth and the shape of the heart. Circulation. 2013;127(2):160-1. doi:10.1161/CIRCULATIONAHA.112.152827.
20. Romero R, Nicolaides K, Conde-Agudelo A, Tabor A, O'Brien J M, Cetingoz E et al. Vaginal progesterone in women with an asymptomatic sonographic short cervix in the midtrimester decreases preterm delivery and neonatal morbidity: a systematic review and metaanalysis of individual patient data. American Journal of Obstetrics & Gynecology. 2012;206(2):124.e1-.e19. doi:10.1016/j.ajog.2011.12.003.
21. Conde-Agudelo A, Romero R, Nicolaides K, Chaiworapongsa T, O'Brien J M, Cetingoz E et al. Vaginal progesterone vs cervical cerclage for the prevention of preterm birth in women with a sonographic short cervix, previous preterm birth, and singleton gestation: a systematic review and indirect comparison metaanalysis. American Journal of Obstetrics & Gynecology. 2013;208(1):42.e1-.e18. doi:10.1016/j.ajog.2012.10.877.
22. Dodd J M, Jones L, Flenady V, Cincotta R, Crowther C A. Prenatal administration of progesterone for preventing preterm birth in women considered to be at risk of preterm birth. Cochrane Database of Systematic Reviews. 2013(7). doi:10.1002/14651858.CD004947.pub3.
23. Nguyen B T, Jensen J T. Evaluating the efficacy and safety of a progestin- and estrogen-releasing ethylene vinyl acetate copolymer contraceptive vaginal ring. Expert Opinion on Drug Safety. 2014;13(10):1423-30. doi:10.1517/14740338.2014.948842.
24. Roumen F J M E, Mishell D R. The contraceptive vaginal ring, NuvaRing®, a decade after its introduction. The European Journal of Contraception & Reproductive Health Care. 2012;17(6):415-27. doi:10.3109/13625187.2012.713535.
25. Brache V, Faundes A. Contraceptive vaginal rings: a review. Contraception. 2010;82(5):418-27. doi:10.1016/j.contraception.2010.04.012.
26. van Laarhoven J A H, Kruft M A B, Vromans H. In vitro release properties of etonogestrel and ethinyl estradiol from a contraceptive vaginal ring. International Journal of Pharmaceutics. 2002;232(1):163-73.
27. Matlin S A, Belenguer A, Hall P E. Progesterone-releasing vaginal rings for use in postpartum contraception. I. In vitro release rates of progesterone from core-loaded rings. Contraception. 1992;45(4):329-41. doi:10.1016/0010-7824(92)90055-X.
28. Nath A, Sitruk-Ware R. Progesterone vaginal ring for contraceptive use during lactation. Contraception. 2010;82(5):428-34. doi:10.1016/j.contraception.2010.05.016.
29. RamaRao S, Clark H, Merkatz R, Sussman H, Sitruk-Ware R. Progesterone vaginal ring: introducing a contraceptive to meet the needs of breastfeeding women. Contraception. 2013;88(5):591-8. doi:10.1016/j.contraception.2013.05.004.
30. Carr S L, Gaffield M E, Dragoman M V, Phillips S. Safety of the progesterone-releasing vaginal ring (PVR) among lactating women: A systematic review. Contraception. 2016;94(3):253-61. doi:10.1016/j.contraception.2015.04.001.
31. Dragonis C, Maltaris T, Binder H, Kat M, Mueller A, Cupisti S et al. Progesterone bioavailability with a progesterone-releasing silicone vaginal ring in IVF candidates. European Journal of Medical Research. 2007;12:264-7.
32. Stadtmauer L, Silverberg K M, Ginsburg E S, Weiss H, Howard B. Progesterone vaginal ring versus vaginal gel for luteal support with invitro fertilization: a randomized comparative study. Fertility and Sterility. 2013;99(6):1543-9. doi:10.1016/j.fertnstert.2012.12.052.
33. Stadtmauer L, Harrison D D, Boyd J, Bocca S, Oehninger S. Pilot study evaluating a progesterone vaginal ring for luteal-phase replacement in donor oocyte recipients. Fertility and Sterility. 2009;92(5):1600-5. doi:10.1016/j.fertnstert.2008.08.085.
34. Stadtmauer L, Waud K. Progesterone Vaginal Ring for Luteal Support. Journal of Obstetrics and Gynaecology of India. 2015;65(1):5-10. doi:10.1007/s13224-014-0634-0.
35. Zegers-Hochschild F, Balmaceda J P, Fabres C, Alam V, Mackenna A, Fernández E et al. Prospective randomized trial to evaluate the efficacy of a vaginal ring releasing progesterone for IVF and oocyte donation. Human Reproduction. 2000;15(10):2093-7. doi:10.1093/humrep/15.10.2093.
36. Romero R, Nicolaides K H, Conde-Agudelo A, O'Brien J M, Cetingoz E, Da Fonseca E et al. Vaginal progesterone decreases preterm birth ≤34 weeks of gestation in women with a singleton pregnancy and a short cervix: an updated meta-analysis including data from the OPPTIMUM study. Ultrasound in Obstetrics & Gynecology. 2016;48(3):308-17. doi:doi:10.1002/uog.15953.
37. Norman J E, Bennett P. Preterm birth prevention—Time to PROGRESS beyond progesterone. PLOS Medicine. 2017;14(9):e1002391. doi:10.1371/journal.pmed.1002391.

All references cited herein are incorporated by reference.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

What is claimed is:

1. An ethylene-vinyl-acetate (EVA), intravaginal ring (IVR), wherein the ring contains at least two segments/fibers, wherein one segment contains progesterone (P) or P equivalent.

2. The EVA ring according to claim 1, further comprising wherein the EVA ring releases about 4 mg/day to about 12 mg/day P after day 1 of insertion.

3. The EVA ring according to claim 2, wherein the EVA ring releases about 4 mg/day P after day 1 of insertion

4. The EVA ring according to claim 1, wherein the EVA ring releases about 8 mg/day P after day 1 of insertion.

5. The EVA ring according to claim 1, wherein the EVA ring releases about 12 mg/day P after day 1 of insertion.

6. The EVA ring according to claims 1-5, wherein the EVA segment containing P is prepared using EVA (about 28% vinyl acetate content) with a final drug loading of about 27% w/w.

7. The EVA ring according to claims 1-6, wherein the EVA segment containing P is about 74.5 mm to about 156.0 mm in length.

8. The EVA ring according to claim 7, wherein the EVA segment containing P is about 74.5mm to about 148.5 mm in length.

9. The EVA ring according to claim 7, wherein the EVA segment containing P is selected from the group consisting of about 74.5 mm, about 148.5 mm and about 156.0 mm.

10. The EVA ring according to claims 1-9, wherein the ring is about 57 mm in diameter with a cross-section diameter of about 5 mm.

11. The EVA ring according to claim 2, wherein the rate of release of P is from about 4 mg/day, about 8 mg/day or about 12 mg/day days over a period of about 2 days through about 14 days.

12. The EVA ring according to claim 11, wherein the release rates for the 4 mg/day progesterone segmented IVR is about 20.5±2.2 for 0-24 hours; about 3.8±1.3 for days 2-14, and about 2.3±1.5 on day 14.

13. The EVA ring according to claim 11, wherein the release rates for the 8 mg/day progesterone segmented IVR is about 38.4±3.3 for 0-24 hours; about 7.4±2.4 for days 2-14, and about 4.7±1.9 on day 14.

14. The EVA ring according to claim 11, wherein the release rates for the 12 mg/day progesterone segmented IVR is about 60.5±2.5 for 0-24 hours; about 11.5±2.8 for days 2-14, and about 7.3±3.8 on day 14.

15. The EVA ring of claim 3, further comprising wherein the Cₘₐₓ (pg/ml) for the 4 mg/day progesterone segmented IVR is about 969±145.

16. The EVA ring of claim 15, further comprising wherein the AUC₀₋₃₃₆ₕ pg h/ml is about 153,000±38,900.

17. The EVA ring of claim 16, further comprising wherein the C_{AVG} (pg/ml) is about 455±116.

18. The EVA ring of claims 17, further comprising wherein the Tₘₐₓ (h) is about 12.

19. The EVA ring of claim 4, further comprising wherein the Cₘₐₓ (pg/ml) for the 8 mg/day progesterone segmented IVR is about 1,820±469.

20. The EVA ring of claim 19, further comprising wherein the AUC₀₋₃₃₆ₕ is 229,000±40,700.

21. The EVA ring of claim 20, further comprising wherein the C_{AVG} is about 682±121.

22. The EVA ring of claim 21, further comprising wherein the Tₘₐₓ is about 2.

23. The EVA ring of claim 5, further comprising wherein the Cₘₐₓ (pg/ml) for the 12 mg/day progesterone segmented IVR is about 2,520±432.

24. The EVA ring of claim 23, further comprising wherein the AUC₀₋₃₃₆ₕ (pg h/ml) is about 350,000±73,900.

25. The EVA ring of claim 24, further comprising wherein the C_{AVG} (pg/ml) is about 1,040±220.

26. The EVA ring of claim 25, further comprising wherein the Tₘₐₓ (h) is about 4.

27. The EVA ring of claims 1-26, wherein the rings are used to prevent preterm birth in which the subject being treated has a shortened cervix.

28. The EVA ring of claims 1-26, wherein the rings are used to treat or ameliorate luteal phase deficiency or as luteal phase support.

29. The EVA ring of claim 28, wherein the luteal phase support is used with assisted reproductive technologies.

30. The EVA ring of claim 29, wherein the luteal phase deficiency is associated with low follicular-stimulating hormone (FSH) levels, altered FSH/luteinizing hormone (LH) ratio, or abnormal FSH and LH pulsatility.

31. The EVA ring of claim 30, wherein the abnormal FSH and LH pulsatility is caused by hypothalamic amenorrhea, thyroid and prolactin disorders, obesity and polycystic ovary syndrome (PCOS) and controlled ovarian stimulation (COS) for IVF cycles.

32. The EVA ring of claims 1-25, wherein the ring is used to cause menstrual periods in women who have not reached menopause but are not having periods due to lack of progesterone in the body, or to prevent overgrowth in the lining of the uterus in postmenopausal women receiving estrogen replacement therapy.
